SEQUENCE LISTING (11 sequences)

SEQ ID NO: 1 - protein, 1148 aa, Bacillus thuringiensis
  Cry1Ea (1)..(1148)
MEIVNNQNQCVPYNCLNNPENEILDIERSNSTVATNIALEISRLLASATPIGGILLGLFDAIWGSIGPSQWDLFLEQIELLIDQKIEEFARNQAISRLEGISSLYGIYTEAFREWEADPTNPALKEEMRTQFNDMNSILVTAIPLFSVQNYQVPFLSVYVQAANLHLSVLRDVSVFGQAWGFDIATINSRYNDLTRLIPIYTDYAVRWYNTGLDRLPRTGGLRNWARFNQFRRELTISVLDIISFFRNYDSRLYPIPTSSQLTREVYTDPVINITDYRVGPSFENIENSAIRSPHLMDFLNNLTIDTDLIRGVHYWAGHRVTSHFTGSSQVITTPQYGITANAEPRRTIAPSTFPGLNLFYRTLSNPFFRRSENITPTLGINVVQGVGFIQPNNAEVLYRSRGTVDSLNELPIDGENSLVGYSHRLSHVTLTRSLYNTNITSLPTFVWTHHSATNTNTINPDIITQIPLVKGFRLGGGTSVIKGPGFTGGDILRRNTIGEFVSLQVNINSPITQRYRLRFRYASSRDARITVAIGGQIRVDMTLEKTMEIGESLTSRTFSYTNFSNPFSFRANPDIIRIAEELPIRGGELYIDKIELILADATLEAESDLERAQKAVNALFTSSNQIGLKTDVTDYHIDRVSNLVECLSDEFCLDEKKELSEKVKHAKRLSDERNLLQDPNFRGINRQLDRGWRGSTDITIQGGDDVFKENYVTLLGTFDECYPTYLYQKIDESKLKAYTRYQLRGYIEDSQDLEIYLIRYNAKHETVNVPGTGSLWPLSAPSPIGKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDRLQADTNIAMIHAADKRVHSIREAYLPELSVIPGVNAAIFEELEGRIFTAFSLYDARNVIKNGDFNNGLSCWNVKGHVDVEEQNNHRSVLVVPEWEAEVSQEVRVCPGRGYILRVTAYKEGYGEGCVTIHEIENNTDELKFSNCVEEEVYPNNTVTCNDYTATQEEYEGTYTSRNRGYDGAYESNSSVPADYASAYEEKAYTDGRRDNPCESNRGYGDYTPLPAGYVTKELEYFPETDKVWIEIGETEGTFIVDSVELLLMEE

SEQ ID NO: 2 - protein, 1155 aa, Bacillus thuringiensis
  Cry1Ab (1)..(1155)
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTLEAESDLERAQKAVNALFTSSNQIGLKTDVTDYHIDRVSNLVECLSDEFCLDEKKELSEKVKHAKRLSDERNLLQDPNFRGINRQLDRGWRGSTDITIQGGDDVFKENYVTLLGTFDECYPTYLYQKIDESKLKAYTRYQLRGYIEDSQDLEIYLIRYNAKHETVNVPGTGSLWPLSAPSPIGKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDRLQADTNIAMIHAADKRVHSIREAYLPELSVIPGVNAAIFEELEGRIFTAFSLYDARNVIKNGDFNNGLSCWNVKGHVDVEEQNNHRSVLVVPEWEAEVSQEVRVCPGRGYILRVTAYKEGYGEGCVTIHEIENNTDELKFSNCVEEEVYPNNTVTCNDYTATQEEYEGTYTSRNRGYDGAYESNSSVPADYASAYEEKAYTDGRRDNPCESNRGYGDYTPLPAGYVTKELEYFPETDKVWIEIGETEGTFIVDSVELLLMEE

SEQ ID NO: 3 - protein, 1186 aa, Bacillus thuringiensis
  Cry1Be (1)..(1186)
MTSNRKNENEIINALSIPAVSNHSAQMNLSTDARIEDSLCIAEGNNIDPFVSASTVQTGINIAGRILGVLGVPFAGQIASFYSFLVGELWPRGRDPWEIFLEHVEQLIRQQVTENTRDTALARLQGLGNSFRAYQQSLEDWLENRDDARTRSVLYTQYIALELDFLNAMPLFAIRNQEVPLLMVYAQAANLHLLLLRDASLFGSEFGLTSQEIQRYYERQVEKTREYSDYCARWYNTGLNNLRGTNAESWLRYNQFRRDLTLGVLDLVALFPSYDTRVYPMNTSAQLTREIYTDPIGRTNAPSGFASTNWFNNNAPSFSAIEAAVIRPPHLLDFPEQLTIFSVLSRWSNTQYMNYWVGHRLESRTIRGSLSTSTHGNTNTSINPVTLQFTSRDVYRTESFAGINILLTTPVNGVPWARFNWRNPLNSLRGSLLYTIGYTGVGTQLFDSETELPPETTERPNYESYSHRLSNIRLISGNTLRAPVYSWTHRSADRTNTISSDSITQIPLVKSFNLNSGTSVVSGPGFTGGDIIRTNVNGSVLSMGLNFNNTSLQRYRVRVRYAASQTMVLRVTVGGSTTFDQGFPSTMSANESLTSQSFRFAEFPVGISASGSQTAGISISNNAGRQTFHFDKIEFIPITATLEAESDLERAQKAVNALFTSSNQIGLKTDVTDYHIDRVSNLVECLSDEFCLDEKKELSEKVKHAKRLSDERNLLQDPNFRGINRQLDRGWRGSTDITIQGGDDVFKENYVTLLGTFDECYPTYLYQKIDESKLKAYTRYQLRGYIEDSQDLEIYLIRYNAKHETVNVPGTGSLWPLSAPSPIGKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDRLQADTNIAMIHAADKRVHSIREAYLPELSVIPGVNAAIFEELEGRIFTAFSLYDARNVIKNGDFNNGLSCWNVKGHVDVEEQNNHRSVLVVPEWEAEVSQEVRVCPGRGYILRVTAYKEGYGEGCVTIHEIENNTDELKFSNCVEEEVYPNNTVTCNDYTATQEEYEGTYTSRNRGYDGAYESNSSVPADYASAYEEKAYTDGRRDNPCESNRGYGDYTPLPAGYVTKELEYFPETDKVWIEIGETEGTFIVDSVELLLMEE

SEQ ID NO: 4 - protein, 1164 aa, Bacillus thuringiensis
  Cry1Ca (1)..(1164)
MDNNPNINECIPYNCLSNPEEVLLDGERISTGNSSIDISLSLVQFLVSNFVPGGGFLVGLIDFVWGIVGPSQWDAFLVQIEQLINERIAEFARNAAIANLEGLGNNFNIYVEAFKEWEEDPKNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGQLTREVYTDPLINFNPQLQSVAQLPTFNVMENSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSATLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATLEAESDLERAQKAVNALFTSSNQIGLKTDVTDYHIDRVSNLVECLSDEFCLDEKKELSEKVKHAKRLSDERNLLQDPNFRGINRQLDRGWRGSTDITIQGGDDVFKENYVTLLGTFDECYPTYLYQKIDESKLKAYTRYQLRGYIEDSQDLEIYLIRYNAKHETVNVPGTGSLWPLSAPSPIGKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDRLQADTNIAMIHAADKRVHSIREAYLPELSVIPGVNAAIFEELEGRIFTAFSLYDARNVIKNGDFNNGLSCWNVKGHVDVEEQNNHRSVLVVPEWEAEVSQEVRVCPGRGYILRVTAYKEGYGEGCVTIHEIENNTDELKFSNCVEEEVYPNNTVTCNDYTATQEEYEGTYTSRNRGYDGAYESNSSVPADYASAYEEKAYTDGRRDNPCESNRGYGDYTPLPAGYVTKELEYFPETDKVWIEIGETEGTFIVDSVELLLMEE

SEQ ID NO: 5 - protein, 1139 aa, Bacillus thuringiensis
  Cry1Da (1)..(1139)
MEINNQNQCVPYNCLSNPKEIILGEERLETGNTVADISLGLINFLYSNFVPGGGFIVGLLELIWGFIGPSQWDIFLAQIEQLISQRIEEFARNQAISRLEGLSNLYKVYVRAFSDWEKDPTNPALREEMRIQFNDMNSALITAIPLFRVQNYEVALLSVYVQAANLHLSILRDVSVFGERWGYDTATINNRYSDLTSLIHVYTNHCVDTYNQGLRRLEGRFLSDWIVYNRFRRQLTISVLDIVAFFPNYDIRTYPIQTATQLTREVYLDLPFINENLSPAASYPTFSAAESAIIRSPHLVDFLNSFTIYTDSLARYAYWGGHLVNSFRTGTTTNLIRSPLYGREGNTERPVTITASPSVPIFRTLSYITGLDNSNPVAGIEGVEFQNTISRSIYRKSGPIDSFSELPPQDASVSPAIGYSHRLCHATFLERISGPRIAGTVFSWTHRSASPTNEVSPSRITQIPWVKAHTLASGASVIKGPGFTGGDILTRNSMGELGTLRVTFTGRLPQSYYIRFRYASVANRSGTFRYSQPPSYGISFPKTMDAGEPLTSRSFAHTTLFTPITFSRAQEEFDLYIQSGVYIDRIEFIPVTATLEAESDLERAQKAVNALFTSSNQIGLKTDVTDYHIDRVSNLVECLSDEFCLDEKKELSEKVKHAKRLSDERNLLQDPNFRGINRQLDRGWRGSTDITIQGGDDVFKENYVTLLGTFDECYPTYLYQKIDESKLKAYTRYQLRGYIEDSQDLEIYLIRYNAKHETVNVPGTGSLWPLSAPSPIGKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDRLQADTNIAMIHAADKRVHSIREAYLPELSVIPGVNAAIFEELEGRIFTAFSLYDARNVIKNGDFNNGLSCWNVKGHVDVEEQNNHRSVLVVPEWEAEVSQEVRVCPGRGYILRVTAYKEGYGEGCVTIHEIENNTDELKFSNCVEEEVYPNNTVTCNDYTATQEEYEGTYTSRNRGYDGAYESNSSVPADYASAYEEKAYTDGRRDNPCESNRGYGDYTPLPAGYVTKELEYFPETDKVWIEIGETEGTFIVDSVELLLMEE

SEQ ID NO: 6 - protein, 788 aa, Bacillus thuringiensis
  VIP3Ab (1)..(788)
MANMNNTKLNARALPSFIDYFNGIYGFATGIKDIMNMIFKTDTGGNLTLDEILKNQQLLNEISGKLDGVNGSLNDLIAQGNLNTELSKEILKIANEQNQVLNDVNNKLDAINTMLHIYLPKITSMLSDVMKQNYALSLQVEYLSKQLKEISDKLDVINVNVLINSTLTEITPAYQRIKYVNEKFEELTFATETTLKVKKDSSPADILDELTELTELAKSVTKNDVDGFEFYLNTFHDVMVGNNLFGRSALKTASELIAKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALVGFEISNDSMTVLKVYEAKLKQNYQVDKDSLSEVIYSDMDKLLCPDQSEQIYYTNNIVFPNEYVITKIDFTKKMKTLRYEVTANSYDSSTGEIDLNKKKVESSEAEYRTLSANNDGVYMPLGVISETFLTPINGFGLQADENSRLITLTCKSYLRELLLATDLSNKETKLIVPPISFISNIVENGNLEGENLEPWIANNKNAYVDHTGGINGTKVLYVHKDGEFSQFVGGKLKSKTEYVIQYIVKGKASIYLKDKKNENSIYEEINNDLEGFQTVTKRFITGTDSSGIHLIFTSQNGEGAFGGNFIISEIRTSEELLSPELIMSDAWVGSQGTWISGNSLTINSNVNGTFRQNLPLESYSTYSMNFTVNGFGKVTVRNSREVLFEKSYPQLSPKDISEKFTTAANNTGLYVELSRSTSGGAINFRDFSIK

SEQ ID NO: 7 - protein, 612 aa, Artificial
  Protease-Processed Cry1Ea Protein
MEIVNNQNQCVPYNCLNNPENEILDIERSNSTVATNIALEISRLLASATPIGGILLGLFDAIWGSIGPSQWDLFLEQIELLIDQKIEEFARNQAISRLEGISSLYGIYTEAFREWEADPTNPALKEEMRTQFNDMNSILVTAIPLFSVQNYQVPFLSVYVQAANLHLSVLRDVSVFGQAWGFDIATINSRYNDLTRLIPIYTDYAVRWYNTGLDRLPRTGGLRNWARFNQFRRELTISVLDIISFFRNYDSRLYPIPTSSQLTREVYTDPVINITDYRVGPSFENIENSAIRSPHLMDFLNNLTIDTDLIRGVHYWAGHRVTSHFTGSSQVITTPQYGITANAEPRRTIAPSTFPGLNLFYRTLSNPFFRRSENITPTLGINVVQGVGFIQPNNAEVLYRSRGTVDSLNELPIDGENSLVGYSHRLSHVTLTRSLYNTNITSLPTFVWTHHSATNTNTINPDIITQIPLVKGFRLGGGTSVIKGPGFTGGDILRRNTIGEFVSLQVNINSPITQRYRLRFRYASSRDARITVAIGGQIRVDMTLEKTMEIGESLTSRTFSYTNFSNPFSFRANPDIIRIAEELPIRGGELYIDKIELILADATLEAESDLER

SEQ ID NO: 8 - protein, 591 aa, Artificial
  Protease-Processed Cry1Ab Protein
IETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLER

SEQ ID NO: 9 - protein, 616 aa, Artificial
  Truncated Cry1Be Protein
IEDSLCIAEGNNIDPFVSASTVQTGINIAGRILGVLGVPFAGQIASFYSFLVGELWPRGRDPWEIFLEHVEQLIRQQVTENTRDTALARLQGLGNSFRAYQQSLEDWLENRDDARTRSVLYTQYIALELDFLNAMPLFAIRNQEVPLLMVYAQAANLHLLLLRDASLFGSEFGLTSQEIQRYYERQVEKTREYSDYCARWYNTGLNNLRGTNAESWLRYNQFRRDLTLGVLDLVALFPSYDTRVYPMNTSAQLTREIYTDPIGRTNAPSGFASTNWFNNNAPSFSAIEAAVIRPPHLLDFPEQLTIFSVLSRWSNTQYMNYWVGHRLESRTIRGSLSTSTHGNTNTSINPVTLQFTSRDVYRTESFAGINILLTTPVNGVPWARFNWRNPLNSLRGSLLYTIGYTGVGTQLFDSETELPPETTERPNYESYSHRLSNIRLISGNTLRAPVYSWTHRSADRTNTISSDSITQIPLVKSFNLNSGTSVVSGPGFTGGDIIRTNVNGSVLSMGLNFNNTSLQRYRVRVRYAASQTMVLRVTVGGSTTFDQGFPSTMSANESLTSQSFRFAEFPVGISASGSQTAGISISNNAGRQTFHFDKIEFIPITATLEAESDLER

SEQ ID NO: 10 - protein, 600 aa, Artificial
  Truncated Cry1Ca Protein
ISTGNSSIDISLSLVQFLVSNFVPGGGFLVGLIDFVWGIVGPSQWDAFLVQIEQLINERIAEFARNAAIANLEGLGNNFNIYVEAFKEWEEDPKNPATRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGQLTREVYTDPLINFNPQLQSVAQLPTFNVMENSAIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTHRSATLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATLEAESDLER

SEQ ID NO: 11 - protein, 576 aa, Artificial
  Truncated Cry1Da Protein
LETGNTVADISLGLINFLYSNFVPGGGFIVGLLELIWGFIGPSQWDIFLAQIEQLISQRIEEFARNQAISRLEGLSNLYKVYVRAFSDWEKDPTNPALREEMRIQFNDMNSALITAIPLFRVQNYEVALLSVYVQAANLHLSILRDVSVFGERWGYDTATINNRYSDLTSLIHVYTNHCVDTYNQGLRRLEGRFLSDWIVYNRFRRQLTISVLDIVAFFPNYDIRTYPIQTATQLTREVYLDLPFINENLSPAASYPTFSAAESAIIRSPHLVDFLNSFTIYTDSLARYAYWGGHLVNSFRTGTTTNLIRSPLYGREGNTERPVTITASPSVPIFRTLSYITGLDNSNPVAGIEGVEFQNTISRSIYRKSGPIDSFSELPPQDASVSPAIGYSHRLCHATFLERISGPRIAGTVFSWTHRSASPTNEVSPSRITQIPWVKAHTLASGASVIKGPGFTGGDILTRNSMGELGTLRVTFTGRLPQSYYIRFRYASVANRSGTFRYSQPPSYGISFPKTMDAGEPLTSRSFAHTTLFTPITFSRAQEEFDLYIQSGVYIDRIEFIPVTATLEAESDLER

(19) United States
(12) Patent Application Publication
(10) Pub. No.: US 2014/0109263 A1
(43) Pub. Date: Apr. 17, 2014

(54) USE OF Cry1Ea IN COMBINATIONS FOR MANAGEMENT OF RESISTANT FALL ARMYWORM INSECTS

(71) Applicant: Dow Agrosciences LLC, Indianapolis, IN (US)

(72) Inventors: Joel J. Sheets, Zionsville, IN (US); Kenneth E. Narva, Zionsville, IN (US); Stephanie Burton, Indianapolis, IN (US); Elizabeth A. Caldwell, Indianapolis, IN (US)

(73) Assignee: Dow Agrosciences LLC, Indianapolis, IN (US)

(21) Appl. No.: 14/047,205

(22) Filed: Oct. 7, 2013

Related U.S. Application Data
(60) Provisional application No. 61/710,154, filed on Oct. 5, 2012.

(51) Int. Cl. A01N 63/02; C12N 15/82

(52) U.S. Cl. CPC A01N 63/02; C12N 15/8286
USPC 800/302; 514/4.5; 435/418; 435/91.1; 435/412; 435/415; 435/416

(57) ABSTRACT

The subject invention includes methods and plants for controlling fall armyworm lepidopteran insects, said plants comprising a Cry1Ea insecticidal protein and a second insecticidal protein selected from the group of Cry1Ab, Cry1Be, Cry1Ca, Cry1Da, and Vip3Ab to delay or prevent development of resistance by the insects.

### CROSS REFERENCE

This application claims the benefit of U.S. provisional application 61/710,154, filed on Oct. 5, 2012. The entire disclosure of which is expressly incorporated herein by reference.

### BACKGROUND OF THE INVENTION

Humans grow corn for food and energy applications. Humans also grow many other crops, including soybeans and cotton. Insects eat and damage plants and thereby undermine these human efforts. Billions of dollars are spent each year to control insect pests and additional billions are lost to the damage they inflict. Synthetic organic chemical insecticides have been the primary tools used to control insect pests but biological insecticides, such as the insecticidal proteins derived from Bacillus thuringiensis (Bt), have played an important role in some areas. The ability to produce insect-resistant plants through transformation with Bt insecticidal protein genes has revolutionized modern agriculture and heightened the importance and value of insecticidal proteins and their genes.

Several Bt proteins have been used to create the insect-resistant transgenic plants that have been successfully registered and commercialized to date. These include Cry1Ab, Cry1Ac, Cry1F and Cry3Bb in corn, Cry1Ac and Cry2Ab in cotton, and Cry3A in potato.

The commercial products expressing these proteins express a single protein except in cases where the combined insecticidal spectrum of 2 proteins is desired (e.g., Cry1Ab and Cry3Bb in corn combined to provide resistance to lepidopteran pests and rootworm, respectively) or where the independent action of the proteins makes them useful as a tool for delaying the development of resistance in susceptible insect populations (e.g., Cry1Ac and Cry2Ab in cotton combined to provide resistance management for tobacco budworm). See also U.S. Patent Application Publication No. 2009/0313717, which relates to a Cry2 protein plus a Vip3Aa, Cry1F, or Cry1A for control of Helicoverpa zea or armigerain. WO 2009/132850 relates to Cry1F or Cry1A and Vip3Aa for controlling Spodoptera frugiperda. U.S. Patent Application Publication No. 2008/0311096 relates in part to Cry1Ab for controlling Cry1F-resistant ECB.

That is, some of the qualities of insect-resistant transgenic plants that have led to rapid and widespread adoption of this technology also give rise to the concern that pest populations will develop resistance to the insecticidal proteins produced by these plants. Several strategies have been suggested for preserving the utility of Bt-based insect resistance traits which include deploying proteins at a high dose in combination with a refuge, and alternation with, or co-deployment of, different toxins (McGaughey et al. (1998), “B.t. Resistance Management,” Nature Biotechnol. 16:144-146).

The protein toxins selected for use in an insect resistant management (IRM) stack need to exert their insecticidal effect independently so that resistance developed to one protein does not confer resistance to the second protein (i.e., there is no cross resistance to the proteins). If, for example, a pest population that is resistant to “Protein A” is sensitive to “Protein B”, one would conclude that there is no cross resistance and that a combination of Protein A and Protein B would be effective in delaying resistance to Protein A alone.

In the absence of resistant insect populations, assessments can be made based on other characteristics presumed to be related to mechanism of action and cross-resistance potential. The utility of receptor-mediated binding in identifying insecticidal proteins likely to not exhibit cross resistance has been suggested (van Mellaert et al. 1999). The key predictor of lack of cross resistance inherent in this approach is that the insecticidal proteins do not compete for receptors in a sensitive insect species.

In the event that two Bt toxins compete for the same receptor, then if that receptor mutates in that insect so that one of the toxins no longer binds to that receptor and thus is no longer insecticidal against the insect, it might be the case that the insect will also be resistant to the second toxin (which competitively bound to the same receptor). That is, the insect is said to be cross-resistant to both Bt toxins. However, if two toxins bind to two different receptors, this could be an indication that the insect would not simultaneously develop resistance to those two toxins.

Representative Cry toxins are listed at the website of the official Bt nomenclature committee (Crickmore et al.; lifesci.sussex.ac.uk/home/Neil_Crickmore/Bt/). There are currently nearly 60 main groups of “Cry” proteins (Cry1-Cry59), with additional Cyt proteins and VIP proteins and the like. Many of each numeric group have capital-letter subgroups, and the capital letter subgroups have lower-cased letter sub-subgroups. (Cry1 has A-L, and Cry1A has a-i, for example).

### BRIEF SUMMARY OF THE INVENTION

The subject invention relates in part to the surprising discovery that the insecticidal protein, Cry1Ea, does not compete with Cry1Ab, Cry1Be, Cry1Ca, Cry1Da, or VIP3Ab for binding with fall armyworm (FAW; Spodoptera frugiperda) gut cell membrane receptor preparations. As one skilled in the art will recognize with the benefit of this disclosure, plants that produce any of the subject pairs of proteins (including insecticidal portions of the full-length proteins), which do not competitively bind with each other, can delay or prevent the development of resistance to any of these insecticidal proteins alone.

Thus, the subject invention relates in part to the use of a Cry1Ea protein in combination with a Cry1Ab, Cry1Be, Cry1Ca, Cry1Da, and/or a VIP3Ab protein. Plants (and acreage planted with such plants) that produce Cry1Ea in combination with at least one of the other proteins described herein are included within the scope of the subject invention.

The subject invention also relates in part to triple stacks or “pyramids” of three (or more) toxins, with Cry1Ea being at least one of the proteins in the stack. In some preferred pyramid embodiments, the combination of the selected toxins provides non-cross-resistant action against FAW. Some preferred “three sites of action” pyramid combinations include one of the subject base pair of proteins (Cry1Ea plus Cry1Ab, Cry1Ca, Cry1Da, Vip3Ab, or Cry1Be) and an additional Bt protein for targeting FAW. These particular triple stacks would, according to the subject invention, advantageously and surprisingly provide three sites of action against FAW. This can help to reduce or eliminate the requirement for refuge acreage.

### BRIEF DESCRIPTION OF THE FIGURES

FIG. 1 shows percent specific binding of ¹²⁵I Cry1Ea (0.5 nM) in BBMV's from FAW versus competition by unlabeled homologous Cry1Ea and heterologous Cry1Ab.

FIG. 2 shows the binding of ¹²⁵I Cry1Ab to BBMV's from FAW larvae and its subsequent displacement by increasing concentrations of unlabeled Cry1Ab.

FIG. 3 shows percent specific binding of ¹²⁵I Cry1Ea in BBMV's from FAW versus competition by unlabeled homologous Cry1Ea and heterologous Cry1Ca.

FIG. 4 shows the results of a binding assay in which ¹²⁵I Cry1Ea was bound to BBMV's from FAW larvae and Cry1Ea, Cry1Be, Cry1Da, and VIP3Ab1 ligands were subsequently added.

### BRIEF DESCRIPTION OF THE SEQUENCES

SEQ ID NO: 1 shows the amino acid sequence of an 1148-amino-acid Cry1Ea protein (with a Cry1Ab protoxin segment).

SEQ ID NO: 2 shows the amino acid sequence of a full-length (1155 amino acids) Cry1Ab protein.

SEQ ID NO: 3 shows the amino acid sequence of an 1186-amino-acid Cry1Be protein (with a Cry1Ab protoxin segment).

SEQ ID NO: 4 shows the amino acid sequence of an 1164-amino-acid Cry1Ca protein (with a Cry1Ab protoxin segment).

SEQ ID NO: 5 shows the amino acid sequence of an 1139-amino-acid Cry1Da protein (with a Cry1Ab protoxin segment).

SEQ ID NO: 6 shows the amino acid sequence of a full-length Vip3Ab protein.

SEQ ID NO: 7 shows the amino acid sequence of a protease-processed Cry1Ea protein.

SEQ ID NO: 8 shows the amino acid sequence of a protease-processed Cry1Ab protein.

SEQ ID NO: 9 shows the amino acid sequence of a protease-processed Cry1Be protein.

SEQ ID NO: 10 shows the amino acid sequence of a protease-processed Cry1Ca protein.

SEQ ID NO: 11 shows the amino acid sequence of a protease-processed Cry1Da protein.

### DETAILED DESCRIPTION OF THE INVENTION

The subject invention relates in part to the surprising discovery that Cry1Ea has a unique and independent mode of action and does not compete with Cry1Ab, Cry1Be, Cry1Ca, Cry1Da, or VIP3Ab1 for binding sites in the gut of fall armyworms (FAW; Spodoptera frugiperda). Thus, a Cry1Ea protein can be used in combination with Cry1Ab, Cry1Be, Cry1Ca, Cry1Da, and/or VIP3Ab1 proteins in transgenic corn (and other plants; e.g., cotton, for example) to delay or prevent FAW from developing resistance to these proteins alone. The subject protein, used together with the other Cry or VIP proteins disclosed herein, can be effective at protecting plants (such as maize plants and/or soybean plants) from damage by Cry-resistant fall armyworm. That is, one use of the subject invention is to protect corn and other economically important plant species from damage and yield loss caused by fall armyworm populations that could develop resistance to a single Cry or VIP protein.

The subject invention thus teaches an insect resistant management (IRM) stack comprising Cry1Ea and Cry1Ab, Cry1Be, Cry1Ca, Cry1Da, and/or VIP3Ab1 to prevent or mitigate the development of resistance by FAW to one of these proteins used by itself The present invention includes compositions for controlling FAW, wherein the compositions comprise a Cry1Ea insecticidal protein and a Cry1Ab, Cry1Be, Cry1Ca, Cry1Da, and/or VIP3Ab1 insecticidal protein. The subject compositions include plants and plant cells.

The invention further comprises a host transformed to produce both a Cry1Ea insecticidal protein and a Cry1Ab, Cry1Be, Cry1Ca, Cry1Da, or VIP3Ab1 insecticidal protein, wherein said host is a microorganism or a plant cell. The subject polynucleotide(s) are preferably in a genetic construct under control of a non-Bacillus-thuringiensis promoter(s). The terms “isolated” and “heterologous” connote that the polypeptide or DNA molecules are in a state that is different from their native environment—involving the hand of man. The subject polynucleotides can comprise codon usage for enhanced expression in a plant.

In addition, the invention includes a method of controlling FAWs, wherein the method comprises contacting said FAW or the environment of said FAW with an effective amount of a composition that contains a Cry1Ea core toxin-containing protein pair of the subject invention.

An embodiment of the invention comprises a plant (which can include corn, soybeans, and cotton) comprising plant-expressible genes encoding a Cry1Ea insecticidal protein pair of the subject invention, and seed of such a plant.

A further embodiment of the invention comprises a plant wherein plant-expressible genes encoding a Cry1Ea insecticidal protein pair of the subject invention have been introgressed into said plant, and seed of such a plant.

As described in the Examples, competitive receptor binding studies using radiolabeled Cry1Ea protein and/or radiolabled Cry1Ab protein show that the Cry1Ab, Cry1Be, Cry1Ca, Cry1Da, and VIP3Ab1 proteins do not compete with Cry1Ea for binding in FAW midgut brush border membrane vesicles to which Cry1Ea binds. These results also indicate that the combination of Cry1Ea and these other proteins can be an effective means to mitigate the development of resistance in FAW populations to these proteins. Thus, based in part on the data described herein, it is thought that co-production (stacking) of the Cry1Ea and Cry1Ab, Cry1Ca, Cry1Be, Cry1Da, and/or VIP3Ab1 proteins can be used to produce a high dose IRM stack for controlling/inhibiting/killing FAW.

Other proteins can be added to a subject pair. For example, the subject invention also relates in part to triple stacks or “pyramids” of three (or more) toxins, with a subject pair being the base pair. In some preferred pyramid embodiments, the selected toxins have three separate modes of action against FAW. Some preferred “three modes of action” pyramid combinations include the subject base pair of proteins plus a third protein for targetting FAW. By “separate sites of action,” it is meant any of the given proteins do not cause cross-resistance with each other. These particular triple stacks would, according to the subject invention, advantageously and surprisingly provide three sites of action against FAW. This can help to reduce or eliminate the requirement for refuge acreage.

Thus, one deployment option is to use the subject pair of proteins in combination with a third toxin/gene, and to use this triple stack to mitigate the development of resistance in FAW to any of these toxins. Accordingly, the subject invention also relates in part to triple stacks or “pyramids” of three (or more) toxins. In some preferred pyramid embodiments, the selected toxins have three separate sites of action against FAW.

Included among deployment options of the subject invention would be to use two, three, or more proteins of the subject proteins in crop-growing regions where FAW can develop resistant populations.

Plants (and acreage planted with such plants) that produce any of the subject combinations of proteins are included within the scope of the subject invention. Additional toxins/genes can also be added, but the particular stacks discussed above advantageously and surprisingly provide multiple sites of action against FAW. This can help to reduce or eliminate the requirement for refuge acreage. A field thus planted of over ten acres is thus included within the subject invention.

GENBANK can also be used to obtain the sequences for any of the genes and proteins disclosed or mentioned herein. Relevant sequences are also available in patents. Representative Cry toxins are listed at the website of the official Bt nomenclature committee (Crickmore et al.; lifesci.sussex.ac.uk/home/Neil_Crickmore/Bt/). There are currently about 70 main groups of “Cry” proteins (Cry1-Cry70), with additional VIP proteins and the like.

Combinations of proteins described herein can be used to control lepidopteran pests. Adult lepidopterans, for example, butterflies and moths, primarily feed on flower nectar and are a significant effector of pollination. Nearly all lepidopteran larvae, i.e., caterpillars, feed on plants, and many are serious pests. Caterpillars feed on or inside foliage or on the roots or stem of a plant, depriving the plant of nutrients and often destroying the plant's physical support structure. Additionally, caterpillars feed on fruit, fabrics, and stored grains and flours, ruining these products for sale or severely diminishing their value. As used herein, reference to lepidopteran pests refers to various life stages of the pest, including larval stages.

Some chimeric proteins of the subject invention comprise a full N-terminal core toxin region of a full Bt protein toxin and, at some point past the end of the core toxin portion, the protein has a transition to a heterologous protoxin sequence. The N-terminal, insecticidally active, toxin portion of a Bt protein toxin is referred to as the “core” toxin. The portion that is C-terminal to the core toxin is referred to as the “protoxin” segment or portion. The transition from the core toxin segment to the heterologous protoxin segment can occur at approximately the core toxin/protoxin junction or, in the alternative, a portion of the native protoxin can be retained, with the transition to the heterologous protoxin portion occurring downstream.

As an example, one chimeric protein of the subject invention, is a full core toxin portion of Cry1Ea (roughly the first 600 amino acids) and/or a heterologous protoxin (the remaining amino acids to the C-terminus). In one preferred embodiment, the portion of a chimeric protein comprising the protoxin is derived from a Cry1Ab toxin. Aside from Vip3Ab, all of the proteins for use according to the subject invention share similar full-length and core toxin sizes and structures.

A person skilled in this art will appreciate that Bt protein toxins, even within a certain class such as Cry1Ea, will vary to some extent in length, and the precise location of the transition from core toxin to protoxin will also vary. Typically, the Cry1Ea proteins, for example, are about 1150 to about 1200 amino acids in length. The transition from core toxin portion to protoxin portion will typically occur at between about 50% to about 60% of the full length toxin. The chimeric proteins of the subject invention will include the full expanse of this N-terminal core toxin region. Thus, the chimeric protein will comprise at least about 50% of the full length of the Cry1 Bt toxin protein. This will typically be at least about 590 amino acids. With regard to the protoxin portion, the full expanse of the Cry1Ab protoxin portion extends from the end of the core toxin portion to the C-terminus of the molecule.

Genes and Toxins.

The genes and toxins useful according to the subject invention include not only the full length sequences disclosed but also fragments of these sequences, variants, mutants, and fusion proteins which retain the characteristic insecticidal activity of the core toxins specifically exemplified herein. As used herein, the terms “variants” or “variations” of genes refer to nucleotide sequences which encode the same toxins or which encode equivalent toxins having pesticidal activity. As used herein, the term “equivalent toxins” refers to toxins having the same or essentially the same biological activity against the target pests as the claimed toxins.

As used herein, the boundaries represent approximately 95% (Cry1Ea's), 78% (Cry1E's), and 45% (Cry1's) sequence identity, per “Revision of the Nomenclature for the Bacillus thuringiensis Pesticidal Crystal Proteins,” N. Crickmore, D. R. Zeigler, J. Feitelson, E. Schnepf, J. Van Rie, D. Lereclus, J. Baum, and D. H. Dean. Microbiology and Molecular Biology Reviews (1998) Vol 62: 807-813. These cut offs can also be applied to the core toxins only.

It will be apparent to a person skilled in this art that genes encoding active toxins can be identified and obtained through several means. The specific genes or gene portions exemplified herein may be obtained from the isolates deposited at a culture depository. These genes, or portions or variants thereof, may also be constructed synthetically, for example, by use of a gene synthesizer. Variations of genes may be readily constructed using standard techniques for making point mutations. Also, fragments of these genes can be made using commercially available exonucleases or endonucleases according to standard procedures. For example, enzymes such as Bal31 or site-directed mutagenesis can be used to systematically cut off nucleotides from the ends of these genes. Genes that encode active fragments may also be obtained using a variety of restriction enzymes. Proteases may be used to directly obtain active fragments of these protein toxins.

Fragments and equivalents which retain the pesticidal activity of the exemplified toxins are within the scope of the subject invention. Also, because of the redundancy of the genetic code, a variety of different DNA sequences can encode the amino acid sequences disclosed herein. It is well within the skill of a person trained in the art to create these alternative DNA sequences encoding the same, or essentially the same, toxins. These variant DNA sequences are within the scope of the subject invention. As used herein, reference to “essentially the same” sequence refers to sequences which have amino acid substitutions, deletions, additions, or insertions which do not materially affect pesticidal activity. Fragments of genes encoding proteins that retain pesticidal activity are also included in this definition.

A further method for identifying the genes encoding the toxins and gene portions useful according to the subject invention is through the use of oligonucleotide probes. These probes are detectable nucleotide sequences. These sequences may be detectable by virtue of an appropriate label or may be made inherently fluorescent as described in International Application No. WO93/16094. As is well known in the art, if the probe molecule and nucleic acid sample hybridize by forming a strong bond between the two molecules, it can be reasonably assumed that the probe and sample have substantial homology. Preferably, hybridization is conducted under stringent conditions by techniques well-known in the art, as described, for example, in Keller, G. H., M. M. Manak (1987) DNA Probes, Stockton Press, New York, N.Y., pp. 169-170. Some examples of salt concentrations and temperature combinations are as follows (in order of increasing stringency): 2×SSPE or SSC at room temperature; 1×SSPE or SSC at 42° C.; 0.1×SSPE or SSC at 42° C.; 0.1×SSPE or SSC at 65° C. Detection of the probe provides a means for determining in a known manner whether hybridization has occurred. Such a probe analysis provides a rapid method for identifying toxin-encoding genes of the subject invention. The nucleotide segments which are used as probes according to the invention can be synthesized using a DNA synthesizer and standard procedures. These nucleotide sequences can also be used as PCR primers to amplify genes of the subject invention.

Variant Toxins.

Certain toxins of the subject invention have been specifically exemplified herein. Since these toxins are merely exemplary of the toxins of the subject invention, it should be readily apparent that the subject invention comprises variant or equivalent toxins (and nucleotide sequences coding for equivalent toxins) having the same or similar pesticidal activity of the exemplified toxin. Equivalent toxins will have amino acid homology with an exemplified toxin. This amino acid homology will typically be greater than 75%, preferably be greater than 90%, and most preferably be greater than 95%. The amino acid homology will be highest in critical regions of the toxin which account for biological activity or are involved in the determination of three-dimensional configuration which ultimately is responsible for the biological activity. In this regard, certain amino acid substitutions are acceptable and can be expected if these substitutions are in regions which are not critical to activity or are conservative amino acid substitutions which do not affect the three-dimensional configuration of the molecule. For example, amino acids may be placed in the following classes: non-polar, uncharged polar, basic, and acidic. Conservative substitutions whereby an amino acid of one class is replaced with another amino acid of the same type fall within the scope of the subject invention so long as the substitution does not materially alter the biological activity of the compound. Below is a listing of examples of amino acids belonging to each class.

| Class of Amino Acid | Examples of Amino Acids |
|---|---|
| Nonpolar | Ala, Val, Leu, Ile, Pro, Met, Phe, Trp |
| Uncharged Polar | Gly, Ser, Thr, Cys, Tyr, Asn, Gln |
| Acidic | Asp, Glu |
| Basic | Lys, Arg, His |

In some instances, non-conservative substitutions can also be made. The critical factor is that these substitutions must not significantly detract from the biological activity of the toxin.

Recombinant Hosts.

The genes encoding the toxins of the subject invention can be introduced into a wide variety of microbial or plant hosts. Expression of the toxin gene results, directly or indirectly, in the intracellular production and maintenance of the pesticide. Conjugal transfer and recombinant transfer can be used to create a Bt strain that expresses both toxins of the subject invention. Other host organisms may also be transformed with one or both of the toxin genes then used to accomplish the synergistic effect. With suitable microbial hosts, e.g., Pseudomonas, the microbes can be applied to the situs of the pest, where they will proliferate and be ingested. The result is control of the pest. Alternatively, the microbe hosting the toxin gene can be treated under conditions that prolong the activity of the toxin and stabilize the cell. The treated cell, which retains the toxic activity, then can be applied to the environment of the target pest.

Where the Bt toxin gene is introduced via a suitable vector into a microbial host, and said host is applied to the environment in a living state, it is essential that certain host microbes be used. Microorganism hosts are selected which are known to occupy the “phytosphere” (phylloplane, phyllosphere, rhizosphere, and/or rhizoplane) of one or more crops of interest. These microorganisms are selected so as to be capable of successfully competing in the particular environment (crop and other insect habitats) with the wild-type microorganisms, provide for stable maintenance and expression of the gene expressing the polypeptide pesticide, and, desirably, provide for improved protection of the pesticide from environmental degradation and inactivation.

A large number of microorganisms are known to inhabit the phylloplane (the surface of the plant leaves) and/or the rhizosphere (the soil surrounding plant roots) of a wide variety of important crops. These microorganisms include bacteria, algae, and fungi. Of particular interest are microorganisms, such as bacteria, e.g., genera Pseudomonas, Erwinia, Serratia, Klebsiella, Xanthomonas, Streptomyces, Rhizobium, Rhodopseudomonas, Methylophilius, Agrobactenum, Acetobacter, Lactobacillus, Arthrobacter, Azotobacter, Leuconostoc, and Alcaligenes; fungi, particularly yeast, e.g., genera Saccharomyces, Cryptococcus, Kluyveromyces, Sporobolomyces, Rhodotorula, and Aureobasidium. Of particular interest are such phytosphere bacterial species as Pseudomonas syringae, Pseudomonas fluorescens, Serratia marcescens, Acetobacter xylinum, Agrobactenium tumefaciens, Rhodopseudomonas spheroides, Xanthomonas campestris, Rhizobium melioti, Alcaligenes entrophus, and Azotobacter vinlandii; and phytosphere yeast species such as Rhodotorula rubra, R. glutinis, R. marina, R. aurantiaca, Cryptococcus albidus, C. diffluens, C. laurentii, Saccharomyces rosei, S. pretoriensis, S. cerevisiae, Sporobolomyces roseus, S. odorus, Kluyveromyces veronae, and Aureobasidium pollulans. Of particular interest are the pigmented microorganisms.

A wide variety of methods are available for introducing a Bt gene encoding a toxin into a microorganism host under conditions which allow for stable maintenance and expression of the gene. These methods are well known to those skilled in the art and are described, for example, in U.S. Pat. No. 5,135,867, which is incorporated herein by reference.

Treatment of Cells.

Bacillus thuringiensis or recombinant cells expressing the Bt toxins can be treated to prolong the toxin activity and stabilize the cell. The pesticide microcapsule that is formed comprises the Bt toxin or toxins within a cellular structure that has been stabilized and will protect the toxin when the microcapsule is applied to the environment of the target pest. Suitable host cells may include either prokaryotes or eukaryotes, normally being limited to those cells which do not produce substances toxic to higher organisms, such as mammals. However, organisms which produce substances toxic to higher organisms could be used, where the toxic substances are unstable or the level of application sufficiently low as to avoid any possibility of toxicity to a mammalian host. As hosts, of particular interest will be the prokaryotes and the lower eukaryotes, such as fungi.

The cell will usually be intact and be substantially in the proliferative form when treated, rather than in a spore form, although in some instances spores may be employed.

Treatment of the microbial cell, e.g., a microbe containing the Bt toxin gene or genes, can be by chemical or physical means, or by a combination of chemical and/or physical means, so long as the technique does not deleteriously affect the properties of the toxin, nor diminish the cellular capability of protecting the toxin. Examples of chemical reagents are halogenating agents, particularly halogens of atomic no. 17-80. More particularly, iodine can be used under mild conditions and for sufficient time to achieve the desired results. Other suitable techniques include treatment with aldehydes, such as glutaraldehyde; anti-infectives, such as zephiran chloride and cetylpyridinium chloride; alcohols, such as isopropyl and ethanol; various histologic fixatives, such as Lugol iodine, Bouin's fixative, various acids and Helly's fixative (See: Humason, Gretchen L., Animal Tissue Techniques, W. H. Freeman and Company, 1967); or a combination of physical (heat) and chemical agents that preserve and prolong the activity of the toxin produced in the cell when the cell is administered to the host environment. Examples of physical means are short wavelength radiation such as gamma-radiation and X-radiation, freezing, UV irradiation, lyophilization, and the like. Methods for treatment of microbial cells are disclosed in U.S. Pat. Nos. 4,695,455 and 4,695,462, which are incorporated herein by reference.

The cells generally will have enhanced structural stability which will enhance resistance to environmental conditions. Where the pesticide is in a proform, the method of cell treatment should be selected so as not to inhibit processing of the proform to the mature form of the pesticide by the target pest pathogen. For example, formaldehyde will crosslink proteins and could inhibit processing of the proform of a polypeptide pesticide. The method of treatment should retain at least a substantial portion of the bio-availability or bioactivity of the toxin.

Characteristics of particular interest in selecting a host cell for purposes of production include ease of introducing the Bt gene or genes into the host, availability of expression systems, efficiency of expression, stability of the pesticide in the host, and the presence of auxiliary genetic capabilities. Characteristics of interest for use as a pesticide microcapsule include protective qualities for the pesticide, such as thick cell walls, pigmentation, and intracellular packaging or formation of inclusion bodies; survival in aqueous environments; lack of mammalian toxicity; attractiveness to pests for ingestion; ease of killing and fixing without damage to the toxin; and the like. Other considerations include ease of formulation and handling, economics, storage stability, and the like.

Growth of Cells.

The cellular host containing the Bt insecticidal gene or genes may be grown in any convenient nutrient medium, where the DNA construct provides a selective advantage, providing for a selective medium so that substantially all or all of the cells retain the Bt gene. These cells may then be harvested in accordance with conventional ways. Alternatively, the cells can be treated prior to harvesting.

The Bt cells producing the toxins of the invention can be cultured using standard art media and fermentation techniques. Upon completion of the fermentation cycle the bacteria can be harvested by first separating the Bt spores and crystals from the fermentation broth by means well known in the art. The recovered Bt spores and crystals can be formulated into a wettable powder, liquid concentrate, granules or other formulations by the addition of surfactants, dispersants, inert carriers, and other components to facilitate handling and application for particular target pests. These formulations and application procedures are all well known in the art.

Formulations.

Formulated bait granules containing an attractant and spores, crystals, and toxins of the Bt isolates, or recombinant microbes comprising the genes obtainable from the Bt isolates disclosed herein, can be applied to the soil. Formulated product can also be applied as a seed-coating or root treatment or total plant treatment at later stages of the crop cycle. Plant and soil treatments of Bt cells may be employed as wettable powders, granules or dusts, by mixing with various inert materials, such as inorganic minerals (phyllosilicates, carbonates, sulfates, phosphates, and the like) or botanical materials (powdered corncobs, rice hulls, walnut shells, and the like). The formulations may include spreader-sticker adjuvants, stabilizing agents, other pesticidal additives, or surfactants. Liquid formulations may be aqueous-based or non-aqueous and employed as foams, gels, suspensions, emulsifiable concentrates, or the like. The ingredients may include rheological agents, surfactants, emulsifiers, dispersants, or polymers.

As would be appreciated by a person skilled in the art, the pesticidal concentration will vary widely depending upon the nature of the particular formulation, particularly whether it is a concentrate or to be used directly. The pesticide will be present in at least 1% by weight and may be 100% by weight. The dry formulations will have from about 1-95% by weight of the pesticide while the liquid formulations will generally be from about 1-60% by weight of the solids in the liquid phase. The formulations will generally have from about 10² to about 10⁴ cells/mg. These formulations will be administered at about 50 mg (liquid or dry) to 1 kg or more per hectare.

The formulations can be applied to the environment of the lepidopteran pest, e.g., foliage or soil, by spraying, dusting, sprinkling, or the like.

Plant Transformation.

Some preferred recombinant hosts in which to express the insecticidal proteins of the subject invention are plants. More highly preferred hosts are crop plants commonly used to produce food, feed, fuel, and oils. More highly preferred host crop plants are maize, soy, cotton, and canola. Maize is a highly preferred embodiment. Genes encoding Bt toxin proteins, as disclosed herein, can be inserted into plant cells using a variety of techniques which are well known in the art. For example, a large number of cloning vectors comprising a replication system in Escherichia coli and a marker that permits selection of the transformed cells are available for preparation for the insertion of foreign genes into higher plants. The vectors comprise, for example, pBR322, pUC series, M13 mp series, pACYC184, inter alia. Accordingly, the DNA fragment having the sequence encoding the Bt toxin protein can be inserted into the vector at a suitable restriction site. The resulting plasmid is used for transformation into E. coli. The E. coli cells are cultivated in a suitable nutrient medium, then harvested and lysed. The plasmid is recovered. Sequence analysis, restriction analysis, electrophoresis, and other biochemical-molecular biological methods are generally carried out as methods of analysis. After each manipulation, the DNA sequence used can be cleaved and joined to the next DNA sequence. Each plasmid sequence can be cloned in the same or other plasmids. Depending on the method of inserting desired genes into the plant, other DNA sequences may be necessary. If, for example, the Ti or Ri plasmid is used for the transformation of the plant cell, then at least the right border, but often the right and the left border of the Ti or Ri plasmid T-DNA, has to be joined as the flanking region of the genes to be inserted. The use of T-DNA for the transformation of plant cells has been intensively researched and sufficiently described in EP 120 516, Lee and Gelvin (2008), Hoekema (1985), Fraley et al., (1986), and An et al., (1985), and is well established in the art.

Once the inserted DNA has been integrated in the plant genome, it is relatively stable. The transformation vector normally contains a selectable marker that confers on the transformed plant cells resistance to a biocide or an antibiotic, such as Bialaphos, Kanamycin, G418, Bleomycin, or Hygromycin, inter alia. The individually employed marker should accordingly permit the selection of transformed cells rather than cells that do not contain the inserted DNA.

A large number of techniques are available for inserting DNA into a plant host cell. Those techniques include transformation with T-DNA using Agrobacterium tumefaciens or Agrobacterium rhizogenes as transformation vector, fusion, injection, biolistics (microparticle bombardment), or electroporation as well as other methods including nanoparticle transformation and cell penetrating peptide mediated transformation. See for example WO 2008/148223, WO2009/046384, WO2011/046786, WO2011/126644, WO2012/006439, and WO2012/006443. If Agrobacteria are used for the transformation, the DNA to be inserted has to be cloned into special plasmids, namely either into an intermediate vector or into a binary vector. The intermediate vectors can be integrated into the Ti or Ri plasmid by homologous recombination owing to sequences that are homologous to sequences in the T-DNA. The Ti or Ri plasmid also comprises the vir region necessary for the transfer of the T-DNA. Intermediate vectors cannot replicate themselves in Agrobacteria. The intermediate vector can be transferred into Agrobacterium tumefaciens by means of a helper plasmid (conjugation). Binary vectors can replicate themselves both in E. coli and in Agrobacteria. They comprise a selection marker gene and a linker or polylinker which are framed by the Right and Left T-DNA border regions. They can be transformed directly into Agrobacteria (Holsters et al., 1978). The Agrobacterium used as host cell is to comprise a plasmid carrying a vir region. The vir region is necessary for the transfer of the T-DNA into the plant cell. Additional T-DNA may be contained. The bacterium so transformed is used for the transformation of plant cells. Plant explants can advantageously be cultivated with Agrobacterium tumefaciens or Agrobacterium rhizogenes for the transfer of the DNA into the plant cell. Whole plants can then be regenerated from the infected plant material (for example, pieces of leaf, segments of stalk, roots, but also protoplasts or suspension-cultivated cells) in a suitable medium, which may contain antibiotics or biocides for selection. The plants so obtained can then be tested for the presence of the inserted DNA. No special demands are made of the plasmids in the case of injection and electroporation. It is possible to use ordinary plasmids, such as, for example, pUC derivatives.

The transformed cells can be grown and differentiated into whole fertile plants in the usual manner. They can form germ cells and transmit the transformed trait(s) to progeny plants. Such plants can be grown in the normal manner and crossed with plants that have the same transformed hereditary factors or other hereditary factors. The resulting hybrid individuals have the corresponding phenotypic properties. Plant cells of the subject invention can also be non-totipotent/unable to be reproduced into whole plants. Such cells can include leaf cells, for example. However, the subject invention also includes cells from seeds of the subject invention, which can be reproduced into whole plants.

In a preferred embodiment of the subject invention, plants will be transformed with genes wherein the codon usage has been optimized for plants. See, for example, U.S. Pat. No. 5,380,831, which is hereby incorporated by reference. While some truncated toxins are exemplified herein, it is well-known in the Bt art that 130 kDa-type (full-length) toxins have an N-terminal half that is the core toxin, and a C-terminal half that is the protoxin “tail.” Thus, appropriate “tails” can be used with truncated/core toxins of the subject invention. See e.g. U.S. Pat. No. 6,218,188 and U.S. Pat. No. 6,673,990. In addition, methods for creating synthetic Bt genes for use in plants are known in the art (Stewart and Burgin, 2007). One non-limiting example of a preferred transformed plant is a fertile plant comprising a plant expressible gene encoding a Cry1Ea protein, and further comprising a second plant expressible gene encoding a Cry1Ab, Cry1Be, Cry1Ca, Cry1Da, or VIP3Ab protein. Transfer (or introgression) of the Cry1Ea-pair traits into inbred lines can be achieved by recurrent selection breeding, for example by backcrossing. In this case, a desired recurrent parent is first crossed to a donor inbred (the non-recurrent parent) that carries the appropriate gene(s) for the Cry1Ea-pair traits. The progeny of this cross is then mated back to the recurrent parent followed by selection in the resultant progeny for the desired trait(s) to be transferred from the non-recurrent parent. After three, preferably four, more preferably five or more generations of backcrosses with the recurrent parent with selection for the desired trait(s), the progeny will be heterozygous for loci controlling the trait(s) being transferred, but will be like the recurrent parent for most or almost all other genes (see, for example, Poehlman & Sleper (1995) Breeding Field Crops, 4th Ed., 172-175; Fehr (1987) Principles of Cultivar Development, Vol. 1: Theory and Technique, 360-376).

Insect Resistance Management (IRM) Strategies.

Roush et al., for example, outlines two-toxin strategies, also called “pyramiding” or “stacking,” for management of insecticidal transgenic crops. (The Royal Society. Phil. Trans. R. Soc. Lond. B. (1998) 353, 1777-1786).

On their website, the United States Environmental Protection Agency (epa.gov/oppbppdl/biopesticides/pips/bt_corn_refuge_{—}2006.htm) publishes the following requirements for providing non-transgenic (i.e., non-Bt) refuges (a section of non-Bt crops/corn) for use with transgenic crops producing a single Bt protein active against target pests.
“The specific structured requirements for corn borer-protected Bt (Cry1Ab or Cry1F) corn products are as follows:
Structured refuges: 20% non-Lepidopteran Bt corn refuge in Corn Belt;
   50% non-Lepidopteran Bt refuge in Cotton Belt
Blocks
   Internal (i.e., within the Bt field)
   External (i.e., separate fields within ½ mile (¼ mile if possible) of the Bt field to maximize random mating)
In-field Strips
   Strips must be at least 4 rows wide (preferably 6 rows) to reduce the effects of larval movement”

In addition, the National Corn Growers Association, on their website:
(ncga.com/insect-resistance-management-fact-sheet-bt-corn)
   also provides similar guidance regarding the refuge requirements. For example:
“Requirements of the Corn Borer IRM:
Plant at least 20% of your corn acres to refuge hybrids
In cotton producing regions, refuge must be 50%
Must be planted within ½ mile of the refuge hybrids
Refuge can be planted as strips within the Bt field; the refuge strips must be at least 4 rows wide
Refuge may be treated with conventional pesticides only if economic thresholds are reached for target insect
Bt-based sprayable insecticides cannot be used on the refuge corn
Appropriate refuge must be planted on every farm with Bt corn”
   Similar structured refuge guidelines can be used for FAW-protected Bt crops of the subject invention.

As stated by Roush et al. (on pages 1780 and 1784 right column, for example), stacking or pyramiding of two different proteins each effective against the target pests and with little or no cross-resistance can allow for use of a smaller refuge. Roush suggests that for a successful stack, a refuge size of less than 10% refuge, can provide comparable resistance management to about 50% refuge for a single (non-pyramided) trait. For currently available pyramided Bt corn products, the U.S. Environmental Protection Agency requires significantly less (generally 5%) structured refuge of non-Bt corn be planted than for single trait products (generally 20%).

There are various ways of providing the IRM effects of a refuge, including various geometric planting patterns in the fields (as mentioned above) and in-bag seed mixtures, as discussed further by Roush et al. (supra), and U.S. Pat. No. 6,551,962.

The above percentages, or similar refuge ratios, can be used for the subject double or triple stacks or pyramids. For triple stacks with three sites of action against a single target pest, a goal would be zero refuge (or less than 5% refuge, for example). This is particularly true for commercial acreage—of over 10 acres for example.

All patents, patent applications, provisional applications, and publications referred to or cited herein are incorporated by reference in their entirety to the extent they are not inconsistent with the explicit teachings of this specification.

Unless specifically indicated or implied, the terms “a”, “an”, and “the” signify “at least one” as used herein.

Following are examples that illustrate procedures for practicing the invention. These examples should not be construed as limiting. All percentages are by weight and all solvent mixture proportions are by volume unless otherwise noted. All temperatures are in degrees Celsius.

### EXAMPLES

### Example 1

### ¹²⁵I Labeling of Cry1Ea and Cry1Ab Proteins

Iodination of Cry Toxins.

Full-length Cry1Ea and Cry1Ab proteins (SEQ ID NOs:1 and 2) were cleaved by trypsin to produce activated, insecticidal forms (as represented by SEQ ID NOS:7 and 8). Purified truncated Cry toxins were was iodinated using Iodo-Beads or Iodo-gen (Pierce). Briefly, two Iodo-Beads were washed twice with 500 μl of phosphate buffered saline, PBS (20 mM sodium phosphate, 0.15 M NaCl, pH 7.5), and placed into a 1.5 ml centrifuge tube behind lead shielding. To this was added 100 μl of PBS. In a hood and through the use of proper radioactive handling techniques, 0.5 mCi Na¹²⁵I (17.4 Ci/mg, Lot 0114, Amersham) was added to the PBS solution with the Iodo-Bead. The components were allowed to react for 5 minutes at room temperature, then 2-25 μg of highly pure truncated Cry protein was added to the solution and allowed to react for an additional 3-5 minutes. The reaction was terminated by removing the solution from the iodo-beads and applying it to a 0.5 ml desalting Zeba spin column (InVitrogen) equilibrated in PBS. The iodo-bead was washed twice with 10 μl of PBS each and the wash solution also applied to the desalting column. The radioactive solution was eluted through the desalting column by centrifugation at 1,000×g for 2 min.

Radio-purity of the iodinated Cry proteins was determined by SDS-PAGE, phosphorimaging and gamma counting. Briefly, 2 μl of the radioactive protein was separated by SDS-PAGE. After separation, the gels were dried using a BioRad gel drying apparatus following the manufacturer's instructions. The dried gels were imaged by wrapping them in Mylar film (12 μm thick), and exposing them under a Molecular Dynamics storage phosphor screen (35 cm×43 cm), for 1 hour. The plates were developed using a Molecular Dynamics Storm 820 phosphorimager and the imaged analyzed using ImageQuant™ software. The radioactive band along with areas immediately above and below the band were cut from the gel using a razor blade and counted in a gamma counter. Radioactivity was only detected in the Cry protein band and in areas below the band. No radioactivity was detected above the band, indicating that all radioactive contaminants consisted of smaller protein components than the truncated Cry protein. These components most probably represent degradation products.

### Example 2

### BBMV Preparation Protocol

Preparation and Fractionation of Solubilized BBMV's.

Last instar Spodoptera frugiperda larvae were fasted overnight and then dissected in the morning after chilling on ice for 15 minutes. The midgut tissue was removed from the body cavity, leaving behind the hindgut attached to the integument. The midgut was placed in 9× volume of ice cold homogenization buffer (300 mM mannitol, 5 mM EGTA, 17 mM tris. base, pH 7.5), supplemented with Protease Inhibitor Cocktail¹ (Sigma P-2714) diluted as recommended by the supplier. The tissue was homogenized with 15 strokes of a glass tissue homogenizer. BBMV's were prepared by the MgCl₂ precipitation method of Wolfersberger (1993). Briefly, an equal volume of a 24 mM MgCl₂ solution in 300 mM mannitol was mixed with the midgut homogenate, stirred for 5 minutes and allowed to stand on ice for 15 min. The solution was centrifuged at 2,500×g for 15 min at 4° C. The supernatant was saved and the pellet suspended into the original volume of 0.5-X diluted homogenization buffer and centrifuged again. The two supernatants were combined, centrifuged at 27,000×g for 30 min at 4° C. to form the BBMV fraction. The pellet was suspended into 10 ml homogienization buffer and supplemented to protease inhibitiors and centrifuged again at 27,000×g of r30 min at 4° C. to wash the BBMV's. The resulting pellet was suspended into BBMV Storage Buffer (10 mM HEPES, 130 mM KCl, 10% glycerol, pH 7.4) to a concentration of about 3 mg/ml protein. Protein concentration was determined by using the Bradford method (1976) with bovine serum albumin (BSA) as the standard. Alkaline phosphatase determination was made prior to freezing the samples using the Sigma assay following manufacturer's instructions. The specific activity of this marker enzyme in the BBMV fraction typically increased 7-fold compared to that found in the midgut homogenate fraction. The BBMV's were aliquoted into 250 μl samples, flash frozen in liquid N₂ and stored at −80° C. ¹Final concentration of cocktail components (in μM) are AEBSF (500), EDTA (250 mM), Bestatin (32), E-64 (0.35), Leupeptin (0.25), and Aprotinin (0.075).

### Example 3

### Method to Measure Binding of ¹²⁵I Cry Proteins to BBMV Proteins

Binding of ¹²⁵I Cry Proteins to BBMV's.

To determine the optimal amount of BBMV protein to use in the binding assays, a saturation curve was generated. ¹²⁵I radiolabeled Cry protein (0.5 nM) was incubated for 1 hr. at 28° C. with various amounts of BBMV protein, ranging from 0-500 μg/ml in binding buffer (8 mM NaHPO₄, 2 mM KH₂PO₄, 150 mM NaCl, 0.1% bovine serum albumin, pH 7.4). Total volume was 0.5 ml. Bound ¹²⁵I Cry protein was separated from unbound by sampling 150 μl of the reaction mixture in triplicate from a 1.5 ml centrifuge tube into a 500 μl centrifuge tube and centrifuging the samples at 14,000×g for 6 minutes at room temperature. The supernatant was gently removed, and the pellet gently washed three times with ice cold binding buffer. The bottom of the centrifuge containing the pellet was cut out and placed into a 13×75-mm glass culture tube. The samples were counted for 5 minutes each in the gamma counter. The counts contained in the sample were subtracted from background counts (reaction with out any protein) and was plotted versus BBMV protein concentration. The optimal amount of protein to use was determined to be 0.15 mg/ml of BBMV protein.

To determine the binding kinetics, a saturation curve was generated. Briefly, BBMV's (150 μg/ml) were incubated for 1 hr. at 28° C. with increasing concentrations of ¹²⁵I Cry toxin, ranging from 0.01 to 10 nM. Total binding was determined by sampling 150 μl of each concentration in triplicate, centrifugation of the sample and counting as described above. Non-specific binding was determined in the same manner, with the addition of 1,000 nM of the homologous trypsinized non-radioactive Cry toxin (as represented by SEQ ID NOS:7-11, and non-trypsinized SEQ ID NO:6) added to the reaction mixture to saturate all non-specific receptor binding sites. Specific binding was calculated as the difference between total binding and non-specific binding.

Homologous and heterologous competition binding assays were conducted using 150 μg/ml BBMV protein and 0.5 nM of the ¹²⁵I radiolabeled Cry protein. The concentrations of the competitive non-radiolabeled Cry toxins added to the reaction mixture ranged from 0.045 to 1,000 nM and were added at the same time as the radioactive ligand, to assure true binding competition. Incubations were carried out for 1 hr. at 28° C. and the amount of ¹²⁵I Cry protein bound to its receptor toxin measured as described above with non-specific binding subtracted. One hundred percent total binding was determined in the absence of any competitor ligand. Results were plotted on a semi-logarithmic plot as percent total specific binding versus concentration of competitive ligand added.

### Example 4

### Summary of Results

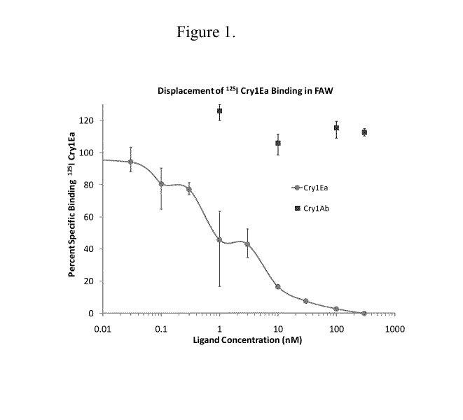

FIG. 1 shows percent specific binding of ¹²⁵I Cry1Ea (0.5 nM) in BBMV's from FAW versus competition by unlabeled homologous Cry1Ea () and heterologous Cry1Ab (▪). The displacement curve for homologous competition by Cry1Ea results in a curve showing 50% displacement of the radioligand at about 1 nM of non-labeled Cry1Ea. Cry1Ab does not displace the specific binding of ¹²⁵I Cry1Ea at any concentration tested, up to 1,000 nM, or 2,000 times the concentration of ¹²⁵I Cry1Ea used in the assay.

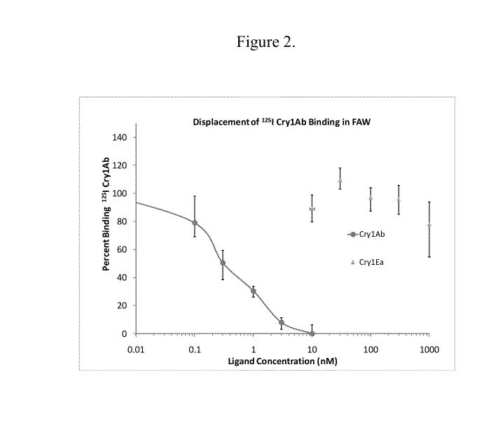

FIG. 2 shows the binding of ¹²⁵I Cry1Ab to BBMV's from FAW larvae and its subsequent displacement by increasing concentrations of unlabeled Cry1Ab. Unlabeled Cry1Ab displaces the binding of the radiolabeled Cry1Ab by 50% at a concentration of about 0.3 NM. The binding of I Cry1Ab to FAW BBMV's is not displaced by Cry1Ea. Thus, these two Cry toxins bind at separate sites in the gut of FAW insects.

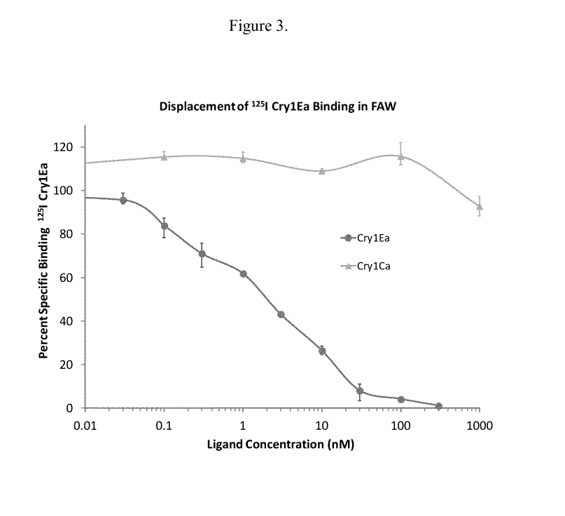

FIG. 3 shows percent specific binding of ¹²⁵I Cry1Ea in BBMV's from FAW versus competition by unlabeled homologous Cry1Ea () and heterologous Cry1Ca (▴). The displacement curve for homologous competition by Cry1Ea results in a curve showing 50% displacement of the radioligand at about 1 nM of non-labeled Cry1Ea. Cry1Ca was not able to displace the binding of ¹²⁵I Cry1Ea.

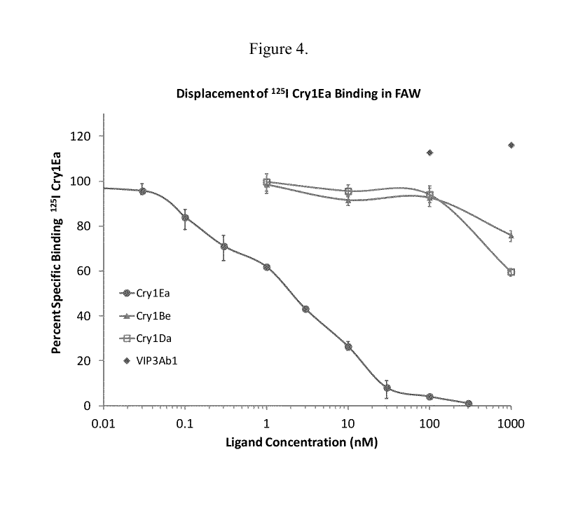

FIG. 4 shows the results of a binding assay in which ¹²⁵I Cry1Ea was bound to BBMV's from FAW larvae and Cry1Ea, Cry1Be, Cry1Da, and VIP3Ab1 ligands were subsequently added. Cry1Be, Cry1Da, and VIP3Ab1 were unable to displace the binding of ¹²⁵I Cry1Ea, except for approximately 40% displacement by Cry1Da at 1,000 nM concentration, or 2,000 times the concentration of ¹²⁵I Cry1Ea used in the assay.

### REFERENCE LIST

Bernardi, R., Tedeschi, G., Ronchi, S., and Palmieri, S. (1996). Isolation and some molecular properties of a trypsin-like enzyme from larvae of European corn borer Ostrinia nubilalis Hnbner (Lepidoptera: pyralidae). Insect Biochemistry and Molecular Biology 26, 883-889.
Christeller, J. T., Laing W. A., Marwick N. P., and Burgess E. P. J. (1992). Midgut protease activities in 12 phytophagous lepidopteran larvae: Dietary and protease inhibitor interactions. Insect Biochem. Molec. Biol. 22, 735-746.
Gatehouse, L. N., Shannon, A. L., Burgess, E. P., and Christeller, J. T. (1997). Characterization of major midgut proteinase cDNAs from Helicoverpa armigera larvae and changes in gene expression in response to four proteinase inhibitors in the diet. Insect Biochem Mol Biol 27, 929-944.
Gomez, I., Arenas, I., Benitez, I., Miranda-Rios, J., Becerril, B., Grande, R., Almagro, J. C., Bravo, A., and Soberon, M. (2006). Specific epitopes of domains II and III of Bacillus thuringiensis Cry1Ab toxin involved in the sequential interaction with cadherin and aminopeptidase-N receptors in Manduca sexta. J. Biol. Chem. 281, 34032-34039.
Gomez, I., Dean, D. H., Bravo, A., and Soberon, M. (2003). Molecular basis for Bacillus thuringiensis Cry1Ab toxin specificity: two structural determinants in the Manduca sexta Bt-R1 receptor interact with loops alpha-8 and 2 in domain II of Cy1Ab toxin. Biochemistry 42, 10482-10489.
Gomez, I., Pardo-Lopez, L., Munoz-Garay, C., Fernandez, L. E., Perez, C., Sanchez, J., Soberon, M., and Bravo, A. (2007). Role of receptor interaction in the mode of action of insecticidal Cry and Cyt toxins produced by Bacillus thuringiensis. Peptides 28, 169-173.
Pigott, C. R. and Ellar, D. J. (2007). Role of receptors in Bacillus thuringiensis crystal toxin activity. Microbiol. Mol. Biol. Rev. 71, 255-281.
Rausell, C., Garcia-Robles, I., Sanchez, J., Munoz-Garay, C., Martinez-Ramirez, A. C., Real, M. D., and Bravo, A. (2004). Role of toxin activation on binding and pore formation activity of the Bacillus thuringiensis Cry3 toxins in membranes of Leptinotarsa decemlineata (Say). Biochim. Biophys Acta 1660, 99-105.
Roush R T. Two-toxin strategies for management of insecticidal transgenic crops: can pyramiding succeed where pesticide mixtures have not? Philos Trans R Soc Lond B Biol Sci. 1998 Oct. 29; 353(1376):1777-1786.
Van, R. J., Jansens, S., Hofte, H., Degheele, D., and Van, M. H. (1990a). Receptors on the brush border membrane of the insect midgut as determinants of the specificity of Bacillus thuringiensis delta-endotoxins. Appl Environ Microbiol 56, 1378-1385.
Heckel, D. G., Gahan, L. J., Baxter, S. W., Zhao, J. Z., Shelton, A. M., Gould, F., and Tabashnik, B. E. (2007). The diversity of Bt resistance genes in species of Lepidoptera. J Invertebr Pathol 95, 192-197.
Luo, K., Banks, D., and Adang, M. J. (1999). Toxicity, binding, and permeability analyses of four bacillus thuringiensis cry1 delta-endotoxins using brush border membrane vesicles of spodoptera exigua and spodoptera frugiperda. Appl. Environ. Microbiol. 65, 457-464.
Palmer, M., Buchkremer, M, Valeva, A, and Bhakdi, S. Cysteine-specific radioiodination of proteins with fluorescein maleimide. Analytical Biochemistry 253, 175-179. 1997. Ref Type: Journal (Full)
Sambrook, J. and Russell, D. W. (2001). Molecular Cloning: A Laboratory Manual. Cold Spring Harbor Laboratory).
Schlenz, M. L., Babcock, J. M., and Storer, N. P. Response of Cry1F-resistant and Susceptible European Corn Borer and Fall Armyworm Colonies to Cry1A.105 and Cry12Ab2. DAI 0830, 2008. Indianapolis, Dow AgroSciences. Derbi Report.
Sheets, J. J. and Storer, N. P. Analysis of Cry1Ac Binding to Proteins in Brush Border Membrane Vesicles of Corn Earworm Larvae (Heleothis zea). Interactions with Cry1F Proteins and Its Implication for Resistance in the Field. DAI-0417, 1-26. 2001. Indianapolis, Dow AgroSciences.
Tabashnik, B. E., Liu, Y. B., Finson, N., Masson, L., and Heckel, D. G. (1997). One gene in diamondback moth confers resistance to four Bacillus thuringiensis toxins. Proc. Natl. Acad. Sci. U.S. A 94, 1640-1644.
Tabashnik, B. E., Malvar, T., Liu, Y. B., Finson, N., Borthakur, D., Shin, B. S., Park, S. H., Masson, L., de Maagd, R. A., and Bosch, D. (1996). Cross-resistance of the diamondback moth indicates altered interactions with domain II of Bacillus thuringiensis toxins. Appl. Environ. Microbiol. 62, 2839-2844.
Tabashnik, B. E., Roush, R. T., Earle, E. D., and Shelton, A. M. (2000). Resistance to Bt toxins. Science 287, 42.
Wolfersberger, M. G. (1993). Preparation and partial characterization of amino acid transporting brush border membrane vesicles from the larval midgut of the gypsy moth (Lymantria dispar). Arch. Insect Biochem. Physiol 24, 139-147.
Xu, X., Yu, L., and Wu, Y. (2005). Disruption of a cadherin gene associated with resistance to Cry1Ac {delta}-endotoxin of Bacillus thuringiensis in Helicoverpa armigera. Appl Environ Microbiol 71, 948-954.

We claim:

1. A transgenic plant comprising a polynucleotide encoding a Cry1Ea insecticidal protein, and a second polynucleotide encoding a second insecticidal protein, said second insecticidal protein being selected from the group consisting of Cry1Ab, Cry1Be, Cry1Ca, Cry1Da, and Vip3Ab.

2. The transgenic plant of claim 1, said plant further comprising DNA encoding a third insecticidal protein.

3. A seed of a plant according to claim 1, wherein said seed comprises said heterologous polynucleotides.

4. A seed of a plant according to claim 3, wherein said seed comprises said heterologous polynucleotides.

5. A field of plants comprising non-Bt refuge plants and a plurality of plants according to claim 1, wherein said refuge plants comprise less than 40% of all crop plants in said field.

6. The field of plants of claim 5, wherein said refuge plants comprise less than 30% of all the crop plants in said field.

7. The field of plants of claim 5, wherein said refuge plants comprise less than 20% of all the crop plants in said field.

8. The field of plants of claim 5, wherein said refuge plants comprise less than 10% of all the crop plants in said field.

9. The field of plants of claim 5, wherein said refuge plants comprise less than 5% of all the crop plants in said field.

10. The field of plants of claim 5, wherein said refuge plants are in blocks or strips.

11. A composition comprising refuge seeds from non-Bt refuge plants, and a plurality of seeds of claim 3, wherein said refuge seeds comprise less than 40% of all the seeds in the composition.

12. The composition of claim 11, wherein said refuge seeds comprise less than 30% of all the seeds in the composition.

13. The composition of claim 11, wherein said refuge seeds comprise less than 20% of all the seeds in the composition.

14. The composition of claim 11, wherein said refuge seeds comprise less than 10% of all the seeds in the composition.

15. The composition of claim 11, wherein said refuge seeds comprise less than 5% of all the seeds in the composition.

16. A method of managing development of resistance to a Cry protein by an insect, said method comprising planting seeds to produce a field of plants of claim 5.

17. A field of any of claims 5-10, wherein said plants occupy more than 10 acres.

18. A plant of claim 1, wherein said plant is selected from the group consisting of corn, soybeans, cotton, canola, and sunflowers.

19. A plant cell of a plant of claim 1, wherein said plant cell comprises said polynucleotide encoding said Cry1Ea insecticidal protein, wherein said Cry1Ea insecticidal protein is at least 95% identical with SEQ ID NO:7.

20. A plant of claim 1 wherein said Cry1Ea insecticidal protein comprises SEQ ID NO:7.

21. A method of producing the polynucleotide of the plant cell of claim 19, said method comprising reproducing said polynucleotide in a plurality of said cells.

22. A method of controlling a fall armyworm insect, said method comprising providing to said insect, for oral ingenstion, a Cry1Ea insecticidal protein and a second insecticidal protein, said second insecticidal protein being selected from the group consisting of Cry1Ab, Cry1Be, Cry1Ca, Cry1Da, and Vip3Ab.

23. A plant cell of a plant of claim 1, wherein said second polynucleotide encodes a Cry1Ab insecticidal protein, and wherein said Cry1Ab insecticidal protein is at least 95% identical with SEQ ID NO:8.

24. A plant of claim 1 wherein said Cry1Ab insecticidal protein comprises SEQ ID NO:8.

25. A plant cell of a plant of claim 1, wherein said plant cell comprises said isolated polynucleotide encoding a Cry1Be insecticidal protein, wherein said Cry1Be insecticidal protein is at least 95% identical with SEQ ID NO:9.

26. A plant of claim 1 wherein said Cry1Be insecticidal protein comprises SEQ ID NO:9.

27. A plant cell of a plant of claim 1, wherein said plant cell comprises said polynucleotide encoding said Cry1Ca insecticidal protein, wherein said Cry1Ca insecticidal protein is at least 95% identical with SEQ ID NO:10.

28. A plant of claim 1 wherein said Cry1Ca insecticidal protein comprises SEQ ID NO:10.

29. A plant cell of a plant of claim 1, wherein said plant cell comprises said polynucleotide encoding said Cry1Da insecticidal protein, wherein said Cry1Da insecticidal protein is at least 95% identical with SEQ ID NO:11.

30. A plant of claim 1 wherein said Cry1Da insecticidal protein comprises SEQ ID NO:11.

31. A plant cell of a plant of claim 1, wherein said plant cell comprises said polynucleotide encoding said Vip3Ab insecticidal protein, wherein said Vip3Ab insecticidal protein is at least 95% identical with SEQ ID NO:6.

32. A plant of claim 1 wherein said Vip3Ab insecticidal protein comprises SEQ ID NO:6.

33. The method of claim 22, said method comprising providing a third insecticidal protein to said insect.

34. A plant cell of the plant of claim 1.

35. A plant cell of the plant of claim 2.

36. A plant cell of the plant of claim 18.
